(19) United States
(12) Patent Application Publication
(10) Pub. No.: US 2020/0315231 A1
(43) Pub. Date: Oct. 8, 2020

(54) CO-CRYSTALLINE SUCROSE

(71) Applicant: NESTES S,A,, Vevey (CH)

(72) Inventors: Heiko Oertling, Lausanne (CH); Thibault Alzieu, Lausanne (CH); Claire Vinay, Saint Marcel-Les-Valence (FR); Celine Borlet, Aigueblanche (FR)

(21) Appl. No.: 16/305,307

(22) PCT Filed: May 30, 2017
(86) PCT No.: PCT/EP2017/063038

§ 371 Date: Nov. 28, 2018

(30) Foreign Application Priority Data
Jun. 2, 2016 (EP) 16172630.2

(51) Int. Cl. A23L 33/125; A23L 33/16; A23L 33/115; A23L 33/17; A61K 31/7016; A61K 33/06; C13B 30/02

(52) U.S. Cl. CPC A23L 33/125; A23L 33/16; A23L 33/115; A61K 9/0053; A61K 31/7016; A61K 33/06; C13B 30/02; A23L 33/17

(57) ABSTRACT

The invention relates to nutritional or pharmaceutical compositions comprising sucrose.calcium salt co-crystals and to the use of sucrose.calcium salt co-crystals for calcium fortification of nutritional compositions. The invention further relates to a process for preparing sucrose.calcium salt co-crystals.

### FIELD OF THE INVENTION

The invention relates to nutritional or pharmaceutical compositions comprising sucrose.calcium salt co-crystals and to the use of sucrose.calcium salt co-crystals for calcium fortification of nutritional compositions. The invention further relates to a process for preparing sucrose.calcium salt co-crystals.

### BACKGROUND OF THE INVENTION

Calcium, the most abundant mineral in the human body, is essential for bone health and teeth development and plays a role in the prevention of developing osteoporosis. Furthermore, calcium is essential in cell physiology, in particular in its role as second messenger, i.e. an intracellular signaling mineral involved in various cellular processes such as proliferation, differentiation, migration and apoptosis. Flux of calcium ions into and out of the cytoplasm functions as a signal for various cellular processes.

Since the body does not produce minerals, it is dependent on an external supply of calcium. An external supply of calcium may for example be provided by fortified nutritional products. Fortification is an increase of the content of essential micronutrients, i.e. vitamins and minerals (e.g. calcium). In this respect, however, selection of an appropriate form of calcium, which supplements the desired level of the mineral without affecting flavour, solubility, bioavailability, processability and organoleptic properties of the product is challenging.

Addition of calcium to milk, for example, is associated with significant difficulties. Direct addition of calcium salts to milk is likely to result in precipitation of calcium complexes of milk proteins. Many potential calcium fortificants are limited in the levels at which they can be applied due to perceived grittiness and bitterness, e.g. calcium sulphate and calcium phosphates. In addition, various calcium salts commonly used for fortification purposes (e.g. calcium citrate malate, tricalcium phosphate or calcium lactate) are characterized by poor flowability rendering their handling and dosage impractical. Many calcium salts absorb moisture from their environment, leading to caking which can block dosing systems and result in loss of entire production batches.

Accordingly, a need exists for solid dosing forms for the mineral calcium, which have good solubility, are flowable and do not absorb moisture and lead to caking, for example in powder formulations.

Thus, it is an object of the present invention to provide an efficient way of calcium fortification in nutritional or pharmaceutical compositions.

Any reference to prior art documents in this specification is not to be considered an admission that such prior art is widely known or forms part of the common general knowledge in the field. As used in this specification, the words “comprises”, “comprising”, and similar words, are not to be interpreted in an exclusive or exhaustive sense. In other words, they are intended to mean “including, but not limited to”.

### SUMMARY OF THE INVENTION

An object of the present invention is to improve the state of the art and to provide compositions overcoming at least some of the inconveniences described above or at least providing a useful alternative. The object of the present invention is achieved by the subject matter of the independent claims. The dependent claims further develop the idea of the present invention.

Accordingly, the present invention provides in a first aspect a nutritional or pharmaceutical composition comprising sucrose.calcium salt co-crystals. A second aspect of the invention relates to the use of sucrose.calcium salt co-crystals for calcium fortification of nutritional compositions. In a third aspect, the invention relates to a process for preparing sucrose.calcium salt co-crystals comprising the steps of preparing a solution comprising a calcium salt and sucrose at a temperature of 70-90° C., cooling the solution to 20-35° C., adding seeding crystals of sucrose.calcium salt co-crystals, allowing the formation of crystals, and isolating the obtained crystals.

It was unexpectedly found that calcium fortification of the nutritional or pharmaceutical compositions is achieved by employing calcium salts in their co-crystalline form with sucrose, offering a novel crystalline, flowable and stable dosing form for supplementary mineralization.

Fructose.calcium halide co-crystals have been reported to be very hygroscopic [Heidar-Ali Tajmir-Riahi, Journal of Inorganic Biochemistry 27, 123-131 (1986)], so the inventors were surprised to find that sucrose.calcium salt co-crystals could be used in formulations, for example powder formulations, without problems of moisture absorption. Sucrose.calcium salt co-crystals have previously been described in the literature [F. T. Jones et al., Microscopy & Crystal Front 13(12), 346-50, (1963)], but their hygroscopic properties have not been examined, nor has their use in nutritional or pharmaceutical compositions been proposed.

### BRIEF DESCRIPTION OF THE DRAWINGS

FIG. 1 shows a single crystal structure elucidation via X-ray diffraction of Sucrose.CaCl₂.4 H₂O.

FIG. 2 shows powder X-ray diffraction patterns of a) top: pure CaCl₂.2 H₂O, b) middle: pure Sucrose and c) bottom: Sucrose.CaCl₂.4 H₂O. The x-axis is 2-theta (in degrees) and the y-axis is intensity (counts).

FIG. 3 shows a single crystal structure elucidation via X-ray diffraction of Sucrose.CaBr₂.4 H₂O.

FIG. 4 shows powder X-ray diffraction patterns of a) top: pure CaBr₂.2 H₂O, b) middle: pure Sucrose and c) bottom: Sucrose.CaBr₂.4 H₂O by slurry ripening in acetone. The x-axis is 2-theta (in degrees) and the y-axis is intensity (counts).

FIG. 5 shows water uptake in maltodextrin powder with the addition of different calcium containing materials: powder matrix alone (♦), powder matrix with CaCl₂.2 H₂O (●), powder matrix with co-crystalline Sucrose.CaCl₂.4 H₂O (▴), and powder matrix with the equivalent dry-mix CaCl₂.2 H₂O+sucrose (▪).

FIG. 6 shows water uptake in skimmed milk powder with the addition of different calcium containing materials: powder matrix alone (♦), powder matrix with CaCl₂.2 H₂O (●), powder matrix with co-crystalline Sucrose.CaCl₂.4 H₂O (▴), and powder matrix with the equivalent dry-mix CaCl₂.2 H₂O+sucrose (▪).

FIG. 7 shows water uptake in full cream milk powder with the addition of different calcium containing materials: powder matrix alone (♦), powder matrix with CaCl₂.2 H₂O (●), powder matrix with co-crystalline Sucrose.CaCl₂.4 H₂O (▴), and powder matrix with the equivalent dry-mix CaCl₂.2 H₂O+sucrose (▪).

FIG. 8 shows water uptake in “growing up milk” powder with the addition of different calcium containing materials: powder matrix alone (♦), powder matrix with CaCl₂.2 H₂O (●), powder matrix with co-crystalline Sucrose.CaCl₂.4 H₂O (▴), and powder matrix with the equivalent dry-mix CaCl₂.2 H₂O+sucrose (▪).

### DETAILED DESCRIPTION OF THE INVENTION

Consequently the present invention relates in part to a nutritional or pharmaceutical composition comprising sucrose.calcium salt co-crystals. “Co-crystals” are crystalline structures comprising at least two components in a defined stoichiometric ratio. For instance the components are atoms, ions or molecules. Crystalline structures have a defined crystalline lattice. The term sucrose.calcium salt co-crystals is used in the context of the current invention to mean sucrose present in co-crystalline form with calcium salt, i.e. the crystalline structure comprises sucrose and a calcium salt. The term co-crystals in the context of the present invention includes multi-component crystalline materials comprised of two or more solids and a liquid. For example, the sucrose.calcium salt co-crystals of the current invention may be sucrose.calcium salt co-crystal hydrates.

It should be noted that calcium saccharate complexes are not sucrose.calcium salt co-crystals. In a saccharate, the sucrose molecule has been deprotonated, for example by the addition of a strong base to the sucrose, leaving a negative charge on the deprotonated sucrose (saccharate).

In the present context, a “nutritional composition” may be any kind of product that provides nutrition to an individual and that may be safely consumed by a human or an animal. A “pharmaceutical composition” as used herein is to be understood as encompassing any pharmaceutically active substance and their salts or/and a pharmaceutical carrier (excipient).

The nutritional or pharmaceutical compositions of the invention may comprise sucrose.calcium salt co-crystals in a concentration greater than 0.01 wt % based on the total weight of the composition, for example in a concentration of 0.01-99 wt % based on the total weight of the composition for example in a concentration of 1-70 wt % based on the total weight of the composition, for further example in a concentration of 5-60 wt % based on the total weight of the composition. In one embodiment, the composition comprises sucrose.calcium salt co-crystals in a concentration of 10-50 wt % based on the total weight of the composition, more preferably in a concentration of 10-20 wt % based on the total weight of the composition.

The nutritional or pharmaceutical composition of the invention may comprise the sucrose.calcium salt co-crystals in a concentration of 0.1-70 wt % based on the total weight of the composition, for example in a concentration of 0.1-50 wt % based on the total weight of the composition, for further example in a concentration of 1-30 wt % based on the total weight of the composition.

An advantage of the invention is that sucrose.calcium salt co-crystals allow for efficient fortification of a food or a beverage with calcium. On a technical scale, additional mineralization of a food product with calcium might raise several issues due to low flowability of current calcium salts commonly used for fortification purposes, which renders handling and dosage difficult. Compositions comprising sucrose.calcium salt co-crystals provide calcium in co-crystalline form combined with sucrose. The co-crystals are characterized by good flowability, and improved processability, resulting in easier handling and dosage. Sucrose.calcium salt co-crystals provide a readily soluble source of calcium, stable in solution. For example, at levels of calcium addition to water where calcium phosphate would show sedimentation, sucrose.calcium salt co-crystals show no sedimentation. This is important for calcium fortification of beverages, especially transparent beverages.

The nutritional or pharmaceutical composition of the invention may further comprise fat, protein or carbohydrates in addition to the sucrose.calcium salt co-crystals.

The calcium salt of the sucrose.calcium salt co-crystals in the composition of the invention may be any non-toxic calcium salt. Preferably the calcium salt of the sucrose.calcium salt co-crystals in the composition of the invention is a material approved for use in nutritional or pharmaceutical products. The calcium salt of the sucrose.calcium salt co-crystals in the composition of the invention may be selected from the group consisting of calcium chloride, calcium bromide, calcium carbonate, calcium hydroxide, calcium acetate, calcium citrate, calcium tartrate, calcium phosphate, di-calcium phosphate, mono-calcium phosphate, calcium pyrophosphates, calcium lactate, calcium malate, calcium citrate malate, and hydrated forms and combinations thereof. For example the calcium salt may be selected from the group consisting of calcium chloride, calcium bromide, calcium phosphate, calcium carbonate and combinations thereof. For further example the calcium salt may be calcium chloride or calcium bromide. The calcium salt may be calcium chloride.

The sucrose.calcium salt co-crystal in the nutritional or pharmaceutical composition of the invention may comprise sucrose and a calcium salt in a molar ratio between 2:1 and 1:2, for example the nutritional or pharmaceutical composition of the invention may comprise sucrose and a calcium salt in equal molar quantities. The sucrose.calcium salt co-crystal in the nutritional or pharmaceutical composition of the invention may be a hydrate, for example the sucrose.calcium salt co-crystal in the nutritional or pharmaceutical composition of the invention may be sucrose.CaCl₂.4 H₂O.

Sucrose.calcium salt co-crystals have a different taste profile compared to the equivalent mixture of sucrose and calcium salt. Forming sucrose and calcium salts into co-crystals can therefore be used to modify the taste of compositions comprising sucrose and calcium salts. High potency sweeteners may advantageously be added to further modify the taste, acting to reinforce the sweetness of the sucrose and helping to counteract any less desirable tastes that may derive from the calcium salts. The nutritional or pharmaceutical composition of the invention may further comprise a high potency sweetener. The high potency sweetener may be selected from the group consisting of abrusoside A, alitame, aspartame, baiyunoside, brazzein, curculin, cyclocarioside I, glycyphyllin, glycyrrhizic acid, a glucosylated steviol glycoside, hernandulcin, N—[N-[3-(3-hydroxy-4-methoxyphenyl)propyl]-L-[alpha]-aspartyl]-L-phenylalanine 1-methyl ester, N—[N-[3-(3-hydroxy-4-methoxyphenyl)-3-methylbutyl]-L-[alpha]-aspartyl]-L-phenylalanine 1-methyl ester, a Luo Han Guo extract, mabinlin, N—[N-[3-(3-methoxy-4-hydroxyphenyl)propyl]-L-[alpha]-aspartyl]-L-phenylalanine 1-methyl ester, monatin, monellin, mukurozioside, neohesperidin dihydrochalcone, neotame, osladin, periandrins, phlomisosides, phloridzin, phyllodulcin, polypodoside A, pterocaryoside A, pterocaryoside B, an ent-kaurane sweetener, thaumatin and trilobatin, and salts and/or solvates thereof.

The nutritional or pharmaceutical composition of the invention may further comprise a nutritive sweetener. For the avoidance of doubt, said nutritive sweetener is in addition to the sucrose comprised within the sucrose.calcium salt co-crystals. The term “nutritive sweetener” as used herein refers to a sweetener that contains carbohydrate and provides energy. Nutritive sweeteners may be further classified into monosaccharides or disaccharides, which impart 4 kcal/g, or sugar alcohols (polyols), which provide an average of 2 kcal/g, as discussed in “Position of the American Dietetic Association: Use of nutritive and nonnutritive sweeteners” J. Am. Diet Assoc. 2004; 104(2):255-275. The nutritional or pharmaceutical composition of the invention may further comprise one or more nutritive sweeteners selected from the group consisting of a 3- to 12-carbon sugar alcohol, a monosaccharide and a sweet disaccharide. For example the nutritional or pharmaceutical composition of the invention may further comprise one or more nutritive sweeteners selected from the group consisting of allose, deoxyribose, erythrulose, galactose, gulose, idose, lyxose, mannose, ribose, tagatose, talose, xylose, erythrose, fuculose, gentiobiose, gentiobiulose, isomaltose, isomaltulose, kojibiose, lactulose, altrose, laminaribiose, arabinose, leucrose, fucose, rhamnose, sorbose, maltulose, mannobiose, mannosucrose, melezitose, melibiose, melibiulose, nigerose, raffinose, rutinose, rutinulose, sophorose, stachyose, threose, trehalose, trehalulose, turanose, xylobiose, sucrose, fructose, glucose, glucose-fructose syrup, high fructose corn syrup, invert sugar, allulose, arabitol, erythritol, glycerol, hydrogenated starch hydrolysate, isomalt, lactitol, maltitol, mannitol, sorbitol and xylitol. The nutritional or pharmaceutical composition of the invention may further comprise a nutritive sweetener selected from the group consisting of sucrose (not in the form of a co-crystal), lactose, glucose and combinations of these.

The nutritional composition of the invention may be selected from the group consisting of a food product; a beverage powder (for example a powder to be reconstituted as a beverage by the addition of water, juice or milk); a composition for clinical nutrition; a food additive or a nutritional supplement. The nutritional composition of the invention may be a food product, for example a confectionery product, an ice cream, a bakery product including cake decorations, a dessert or a pet food product. The nutritional composition of the invention may be a beverage powder, for example a milk suitable for toddlers aged between one year old and three years old such as growing-up milk. Growing-up milks commonly have minerals added to them. The beverage powder may be a powdered creamer such as a coffee creamer.

The nutritional composition of the invention may be a culinary product, for example an instant soup, bouillon cube or bouillon powder, flavouring or powdered cooking aid or dehydrated ready-meal. The nutritional composition of the invention may be a low calorie food product, for example it may have 40 calories or less per reference amounts customarily consumed (RACC) (or per 50 g if the RACC is small) or for a meal or main dish it may have 120 calories or less per 100 g. This is in line with the U.S. Food and Drug Administration definitions of nutrient content claims of January 2013 for a low calorie product.

The nutritional composition of the invention may be a nutritional supplement. Nutritional supplements are nutritional compositions which are provided in addition to a regular diet providing nutrients (macronutrients or micronutrients) or dietary fibres, e.g. micronutrients like certain vitamins, minerals, e.g. macronutrients like fatty acids, amino acids, carbohydrates, protein etc.

Surprisingly, the shelf life of a composition comprising sucrose.calcium salt co-crystals is significantly prolonged in comparison to compositions comprising pure calcium salts. Sucrose.calcium salt co-crystals unexpectedly show an improved moisture tolerance as compared to compositions comprising pure calcium salts.

The pharmaceutical composition of the invention may further comprise pharmaceutically active ingredients and their salts. Pharmaceutically active ingredients are those having direct effect on the cure, mitigation, treatment or prevention of a disease, thereby restoring, enhancing or maintaining physiological functions.

The pharmaceutical composition according to the invention may be administered enterally or parenterally. Enteral administration may be e.g. by mouth, by gastric or duodenal feeding tube or rectally. Parenteral administration may be selected from the group of intravenous, intra-arterial, intra-muscular, intraosseous, intracerebral, intracerebroventricular, intrahecal, subcutaneous administration.

The pharmaceutical composition according to the invention may be orally administrable. Advantageously, sucrose.calcium salt co-crystals are rapidly dissolvable in the consumer's saliva, resulting in a homogeneous, lump-free solution. Due to the ameliorated mouthfeel, consumer acceptance is increased and ingestion is possible for persons suffering from swallowing difficulties, e.g. infants, children or elderly. Further, patients suffering from dysphagia or xerostomia may be treated with the fast-dissolving pharmaceutical compositions of the invention. The orally administrable pharmaceutical composition may be administrable as a tablet, a capsule, a gel capsule, a comprimate, a hard or soft candy, a chewing gum or a pill. For example the tablet may be a buccal, sub-lingual, or orally-disintegrating tablet. The orally administrable pharmaceutical composition may be administrable via a dry-powder inhaler or a nebulizer.

The nutritional or pharmaceutical composition of the invention may be for use in the treatment or prevention of hypocalcemia. Hypocalcemia is the condition of a low level of calcium in the blood. The nutritional or pharmaceutical composition of the invention for use in the treatment or prevention of hypocalcemia may further comprise vitamin D and/or magnesium.

An embodiment the invention provides the use of sucrose.calcium salt co-crystals for calcium fortification of nutritional compositions.

In a further embodiment the invention provides a process for preparing sucrose.calcium salt co-crystals comprising the steps of:
a. preparing a solution comprising a calcium salt and sucrose at a temperature of 70-90° C.,
b. cooling the solution to 20-35° C.,
c. adding seeding crystals of sucrose.calcium salt co-crystals,
d. allowing the formation of crystals,
e. isolating the obtained crystals.

The co-crystals may be prepared by mechanical processes such as grinding, ball milling of a mixture etc. The individual constituents of the respective co-crystal are mixed in the required molar ratio and treated mechanically in standard micronization equipment as for example ball mills, disc mills, planetary ball mills etc. for a certain amount of time. Optionally, a liquid can be added to allow for liquid-assisted grinding (LAG) or formation of stoichiometric solvates, e.g. hydrates or ethanolates.

Optionally, the desired co-crystals can also be produced by established and industrialized techniques such as spray-drying, freeze-drying, twin-screw extrusion, roller-compaction, compression or in certain cases straightforward mechanical mixing/blending.

Those skilled in the art will understand that they can freely combine all features of the present invention disclosed herein. In particular, features described for the product of the present invention may be combined with the method of the present invention and vice versa. Further, features described for different embodiments of the present invention may be combined. Where known equivalents exist to specific features, such equivalents are incorporated as if specifically referred to in this specification.

Further advantages and features of the present invention are apparent from the figures and non-limiting examples.

### EXAMPLES

### Example 1: Synthesis of Seeding Crystals by Direct Evaporation

| | | | Calcium Chloride |
|---|---|---|---|
| | Name | Sucrose | dihydrate |
| ratio | equivalents | 1 | 1.5 |
| 1:1.5 | mass introduced (g) | 5.00 | 3.22 |
| | real mass introduced (g) | 5.00 | 3.21 |
| | amount of substance (mmol) | 14.61 | 21.81 |

### Process:

5 g of sucrose are mixed with 3.21 g of calcium chloride dihydrate in 10 mL of water until complete dissolution. The solution was transferred into a petri-dish to allow complete evaporation. After 2 weeks, the solution began to crystallize. The presence of co-crystalline sucrose.CaCl₂.4 H₂O was confirmed by Powder X-ray diffraction (PXRD) (Example 5). The crystals obtained with this method were used as seeding crystals for the synthesis by cooling (Example 2).

### Example 2: Synthesis of Seeding Crystals Via Cooling

| | | Calcium chloride |
|---|---|---|
| Name | Sucrose | dihydrate |
| equivalents | 1 | 1.5 |
| mass introduced (g) | 41.34 | 26.75 |
| real mass introduced (g) | 41.34 | 26.62 |
| amount of substance (mol) | 0.12083 | 0.18106 |
| amount of substance (mmol) | 120.83 | 181.06 |

In a thermostatted, double-jacketed 200 mL glass reactor equipped with a magnetic stirrer bar, 41.34 g of sucrose (120.83 mmol) and 26.75 g of CaCl₂.2 H₂O (181.06 mmol) were added to a mixture of 25 mL of ethanol and 24 mL of water at ambient temperature. The solution was then heated up to 70° C. for 2 h while stirring at 300 rpm until a clear solution was obtained. After complete dissolution, the solution was cooled down progressively to 18° C. over 4 h.

On standing over two days at 18° C., the solution didn't crystallize, so a seeding with the crystals obtained by evaporation was performed. The suspension was stirred at 18° C. for 2 h and then filtered over a glass frit. The white crystals were dried in the oven at 40° C. for one night to give 32 g product. The yield corresponds to 50%. The characterization (Example 5) confirmed that the product obtained is the co-crystal sucrose.CaCl₂.4 H₂O. This product was used as seeding crystals for the large-scale protocol (Example 3).

### Example 3: Large-Scale Synthesis Protocol

| | | Calcium chloride |
|---|---|---|
| Name | Sucrose | dihydrate |
| equivalents | 1 | 1.5 |
| mass introduced (g) | 661.44 | 428.00 |
| real mass introduced (g) | 661.44 | 425.86 |
| amount of substance (mol) | 1.933 | 2.897 |
| amount of substance (mmol) | 1933 | 2897 |

In a 1 litre double-jacketed, thermostatted glass reactor equipped with overhead stirring, internal temperature control and a water condenser, 661.44 g of sucrose (1.93 mol) and 428 g of CaCl₂.2 H₂O (2.90 mol) were added to a mixture of 400 mL of ethanol and 377.6 mL of water at ambient temperature. The temperature was set to 80° C. and the solution was stirred at 100 rpm during 2 h. After complete dissolution, the solution was cooled down progressively to 25° C. over 5 h. At this temperature, 5 mg of seeding crystals from the previous process were added to the solution. The stirring rate was reduced to 30 rpm and crystallization occurred few minutes after the seeding step. The solution was then cooled down to 18° C. and held at this temperature for 6 h. The solution was filtered over a glass frit and the white crystals were dried in the oven at 40° C. under vacuum for one night to give 474 g of product. The yield corresponds to 47%. The presence of co-crystalline sucrose.CaCl₂.4 H₂O was confirmed by Powder X-ray diffraction (PXRD) (Example 5).

### Example 4: Mechanochemical Synthesis

### Tested Conditions:

| | | | Calcium chloride |
|---|---|---|---|
| | Name | sucrose | dihydrate |
| Ratio | equivalents | 1 | 1 |
| 1:1 | mass introduced (g) | 3.00 | 1.29 |
| | real mass introduced (g) | 3.00 | 1.28 |
| | amount of substance (mmol) | 8.77 | 8.73 |
| ratio | equivalents | 1 | 2 |
| 1:2 | mass introduced (g) | 3.00 | 2.58 |
| | real mass introduced (g) | 3.00 | 2.57 |
| | amount of substance (mmol) | 8.77 | 17.45 |
| ratio | equivalents | 2 | 1 |
| 2:1 | mass introduced (g) | 3.00 | 0.64 |
| | real mass introduced (g) | 3.00 | 0.64 |
| | amount of substance (mmol) | 8.77 | 4.36 |

Three different ratios were tested applying the following conditions during the ball-milling:
No addition of water
Addition of 0.5 equivalents of water
Addition of 2 equivalent of water
Addition of 0.5 equivalent of ethanol
Addition of 0.5 equivalent of acetone
Addition of 0.5 equivalent of isopropanol

### Process:

The two crystalline powders were placed in a Retsch vibratory ball-mill using 5 steel balls of 15 mm diameter. Ball-milling was performed after optional addition of solvent as indicated above for 30 min with a 20 Hz frequency.

### Results:

The co-crystalline sucrose.CaCl₂.4 H₂O was obtained under the following conditions: Two equivalents of Sucrose with one equivalent of CaCl₂.2 H₂O and addition of two equivalents of water. The presence of co-crystalline Sucrose.CaCl₂.4 H₂O was confirmed by Powder X-ray diffraction (PXRD) (Example 5).

### Example 5: Characterization

### X-Ray Diffraction Analysis:

PXRD experiments were carried out with a Rigaku Miniflex 600 diffractometer using CuK_{α} (1.54 Å) radiation with K_{β} filter (Nickel, 3 mm). The detector used was a D/teX Ultra-high-speed 1D. The scanning angle 20 is set to 5-60°, the step size is 0.02°, with a speed of 5° per minute and an operating voltage of 40 kV and amperage of 25 mA.

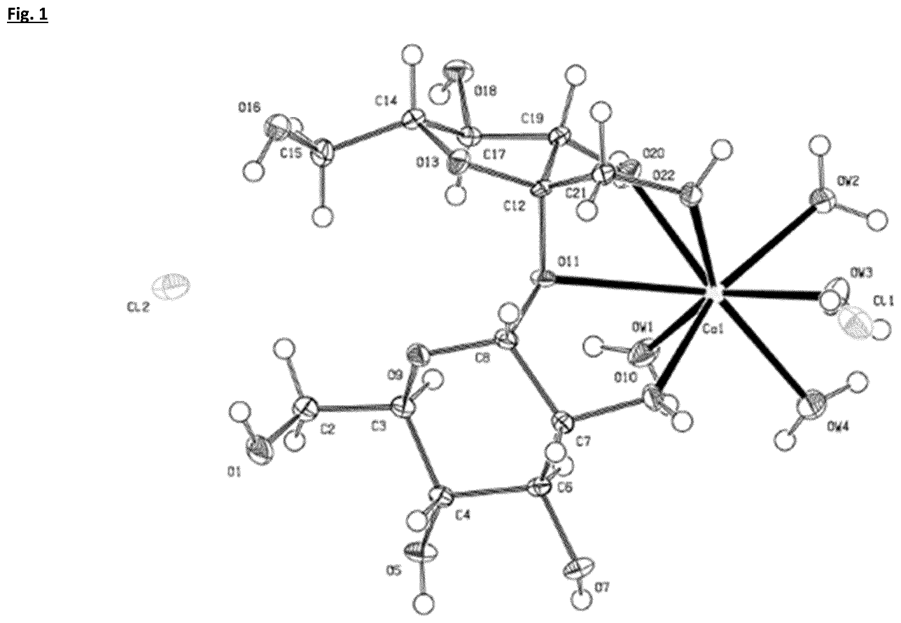

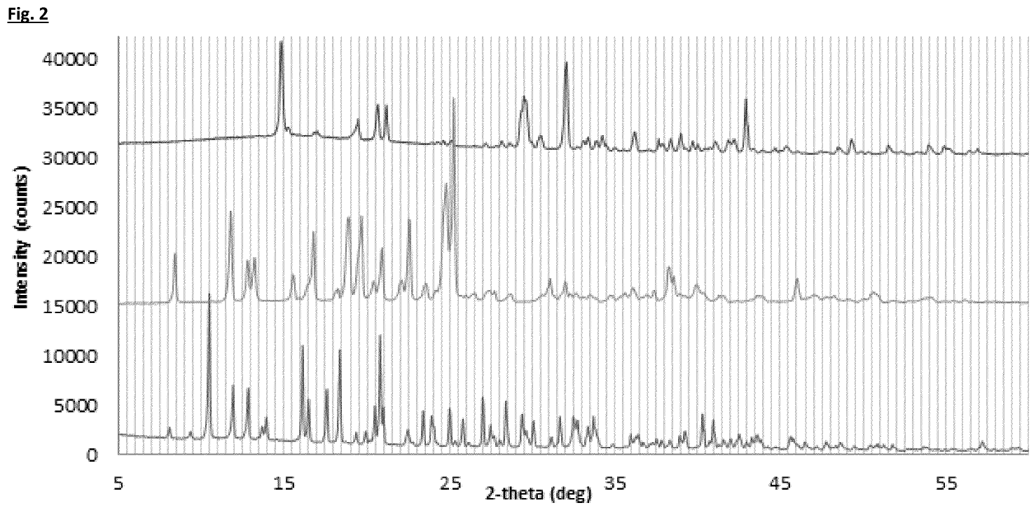

PXRD characterization was performed on the pure components sucrose, CaCl₂.2 H₂O and on the product obtained by crystallization via cooling. As shown in FIG. 2, the powder X-ray diffraction pattern of the co-crystal is clearly different from those of its individual ingredients, which indicates the existence of a new crystalline phase. In order to identify the product formed, single crystals were obtained by slow, direct evaporation from water and the crystalline structure was elucidated via single crystal X-ray diffraction (See FIG. 1). By comparing the unit cell parameters, it could be confirmed that the product formed is indeed the co-crystal sucrose.CaCl₂.4 H₂O described by Jones, Rorem and Palmer in 1963.

Notably, one can state that the calcium ion is coordinated by one sucrose molecule only, and not, as in most other reported carbohydrate calcium co-crystal structures, by two sugars simultaneously. Therefore, the structure is best described as a discrete complex and not as a network or chain-like assembly in the crystalline state. Moreover, the central calcium ion is coordinated by eight oxygen atoms in total, among them four of the associated hydrate water. Interestingly, the glucose-, as well as the fructose-moiety of the sucrose molecule are attached to the central atom, as well as the bridging oxygen atom linking both sugar subunits. The fructose unit is coordinating with two neighboring alcohol oxygen atoms, whereas the six-membered glucose-ring is attached via one alcohol oxygen atom only.

### Example 6: Synthesis of Sucrose.CaBr₂.4 H₂O

Different methods were tested to synthesize this co-crystalline phase. First, ball-milling three different molar ratios of starting materials (1/1, 2/1 and 1/2) under the following conditions was attempted and conducted as described previously:
Addition of 0.5 equivalent of water
Addition of 2 equivalents of water
Addition of 2 equivalents of water and 0.5 equivalent of ethanol
Addition of 2 equivalents of water and 0.5 equivalent of acetone
Addition of 2 equivalents of water and 0.5 equivalent of isopropanol

The outcome were physical mixtures for all conditions tested, except for the equimolar case and addition of 2 equivalents of water and 0.5 equivalent of acetone or isopropanol. New peaks in the PXRDs were observed in those cases.

Experiments to synthesize the bromide-version of the Calcium/Sucrose co-crystal via cooling of an equimolar aqueous solution were not successful, even after adding ethanol as anti-solvent.

Further solution-based experiments followed the so-called “slurry ripening” methodology: the two starting materials were mixed at ambient temperature in acetone (or alternatively isopropanol) in such a fashion that complete dissolution was not effectuated. The resulting slurry or suspension is then stirred for one entire day. The choice of solvents was inspired by the positive results obtained by ball milling. After filtration, new peaks were observable for the acetone-slurry and the PXRD is identical to the pattern obtained in case of a mechanochemical transformation in the presence of 2 equivalents of water and 0.5 equivalent of acetone. However, a physical mixture was obtained in the case of isopropanol.

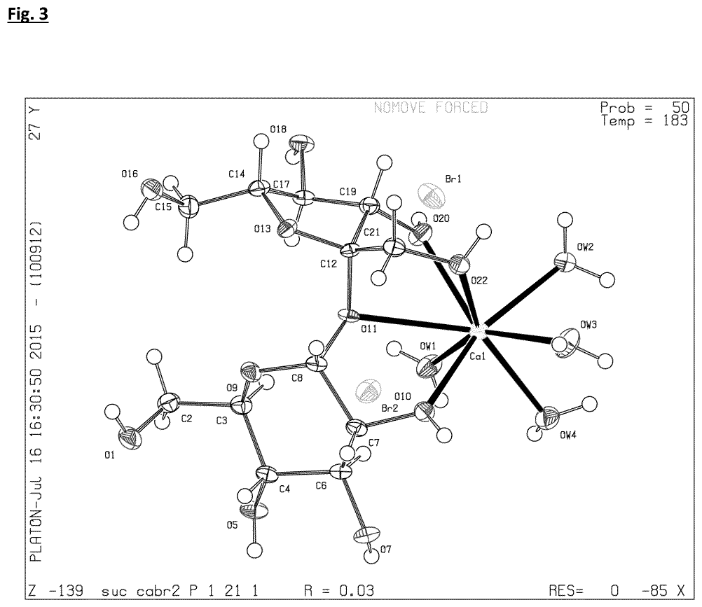

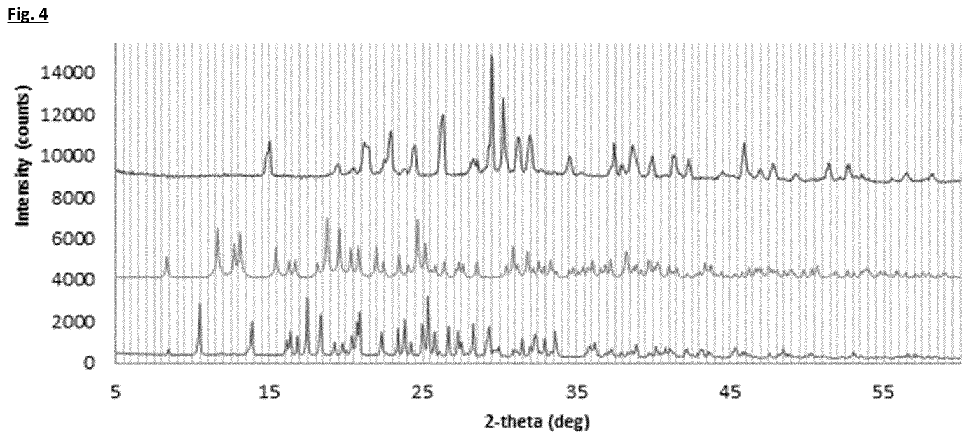

Ultimately, experiments to obtain the pure co-crystalline material by direct evaporation from water were fruitful: the solutions prepared with the molar ratio (1/1) and (1/2), e.g. Sucrose/CaBr₂, crystallized within several days upon standing at ambient temperature and gave crystals prone to single-crystal X-ray analysis. The resulting structure confirmed the formula Sucrose.CaBr₂.4 H₂O (see FIGS. 3 and 4) and is furthermore isostructural to the system Sucrose.CaCl₂.4 H₂O. Space group and unit cell parameters were determined as: monoclinic, a=10.07, b=10.17, c=11.53 Å, α=90.0, β=106.1, γ=90.0°.

### Example 7: Water Uptake Behavior Study for the Co-Crystal of Sucrose.CaCl₂.4 H₂O

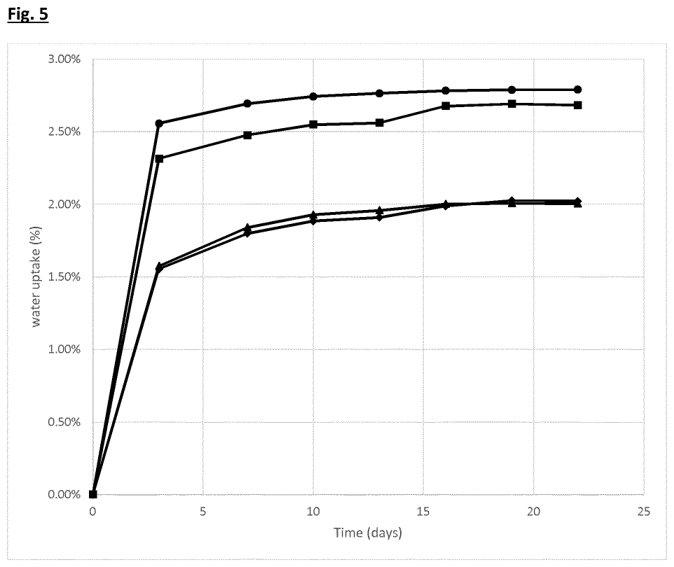

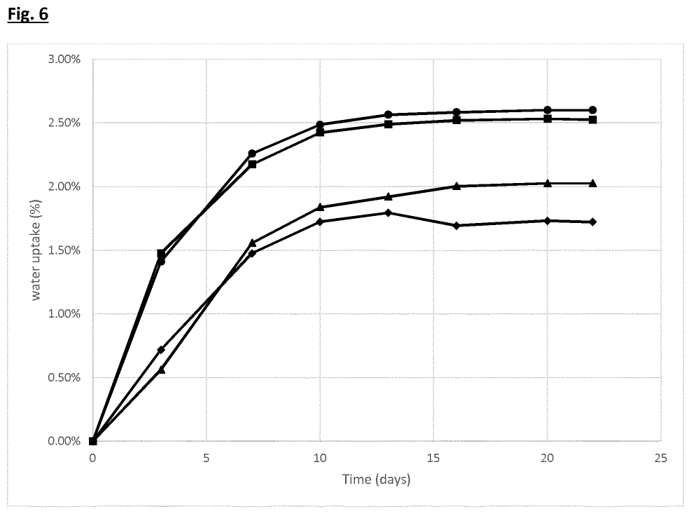

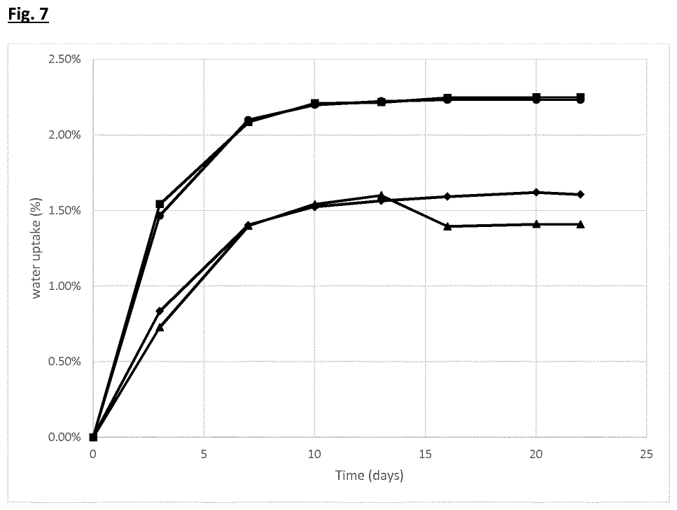

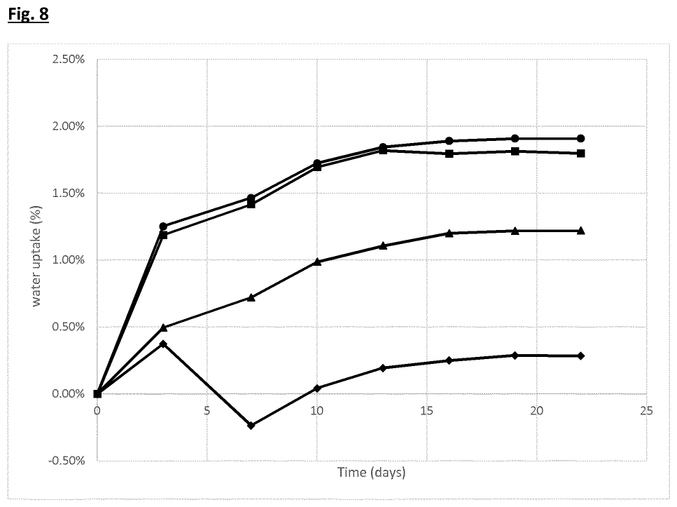

The water uptake behavior was studied for the co-crystal of Sucrose.CaCl₂.4 H₂O in four different powder matrices: Maltodextrin (FIG. 5), skimmed milk powder (FIG. 6), full cream milk powder with 26% fat (FIG. 7), and powdered “growing up milk” (Nestlé BEBA3) (FIG. 8). For each matrix, three different calcium containing material were mixed into the powder to simulate calcium fortification. Powders were stored in a desiccator at 33% Relative Humidity over 22 days. The water uptake was measured every 3-4 days by tracking the weight change. The water uptake is shown in FIGS. 5 to 8): powder matrix alone (♦), powder matrix with CaCl₂. 2 H₂O (2.1% by weight): 13.59 g of matrix+290 mg of CaCl₂.2 H₂O (●), powder matrix with co-crystalline Sucrose.CaCl₂.4 H₂O (7.6% by weight): 13.59 g of matrix+1.04 g of co-crystal (▴), and powder matrix with the equivalent dry-mix: 13.59 g of matrix+290 mg of CaCl₂.2 H₂O+670 mg of sucrose (▪).

Water uptake can be seen to be lower for the co-crystal than for the dry mix in all the samples tested. For maltodextrin, skimmed milk powder and full cream milk powder, the water uptake of the co-crystal is similar to that of the unfortified powder.

1. Nutritional or pharmaceutical composition comprising sucrose.calcium salt co-crystals.

2. A nutritional or pharmaceutical composition according to claim 1 wherein the composition comprises the sucrose.calcium salt co-crystals in a concentration of greater than 0.01 wt % based on the total weight of the composition.

3. A nutritional or pharmaceutical composition according to claim 1 wherein the composition further comprises a component selected from the group consisting of fat, protein and carbohydrates in addition to the sucrose.calcium salt co-crystals.

4. A nutritional or pharmaceutical composition according to claim 1 wherein the calcium salt of the sucrose.calcium salt co-crystals is selected from the group consisting of calcium chloride, calcium bromide, calcium carbonate, calcium hydroxide, calcium acetate, calcium citrate, calcium tartrate, calcium phosphate, di-calcium phosphate, mono-calcium phosphate, calcium pyrophosphates, calcium lactate, calcium malate, calcium citrate malate, and hydrated forms and combinations thereof.

5. A nutritional or pharmaceutical composition according to claim 1 wherein the sucrose.calcium salt co-crystal is sucrose.CaCl₂.4 H₂O.

6. A nutritional or pharmaceutical composition according to claim 1 wherein the composition further comprises a high potency sweetener.

7. A nutritional or pharmaceutical composition according to claim 1 wherein the composition further comprises a nutritive sweetener.

8. A nutritional composition according to claim 1 wherein the nutritional composition is selected from the group consisting of a food product, a beverage powder, a composition for clinical nutrition, a food additive and a nutritional supplement.

9. A pharmaceutical composition according to claim 1, wherein the pharmaceutical composition further comprises pharmaceutically active ingredients and their salts.

10. A pharmaceutical composition according to claim 9, wherein the composition is orally administrable.

11. A method for calcium fortification of nutritional compositions comprising the step of adding use of sucrose.calcium salt co-crystals to a nutritional composition.

12. Process for preparing sucrose.calcium salt co-crystals comprising the steps of:
preparing a solution comprising a calcium salt and sucrose at a temperature of 70-90° C.;
cooling the solution to 20-35° C.;
adding seeding crystals of sucrose.calcium salt co-crystals;
allowing the formation of crystals; and
isolating the obtained crystals.
